(19) United States
(12) Patent Application Publication
(10) Pub. No.: US 2024/0423616 A1
(43) Pub. Date: Dec. 26, 2024

(54) COMPRESSIBLE SURGICAL ADJUNCTS, CARTRIDGES, AND CARTRIDGE ASSEMBLIES

(71) Applicant: CILAG GMBH INTERNATIONAL, ZUG (CH)

(72) Inventors: PEYTON HOPSON, CINCINNATI, OH (US); MADELAINE FRANZONI, CINCINNATI, OH (US); CHRISTOPHER Q. SEOW, CINCINNATI, OH (US); MICHAEL J. VENDELY, LEBANON, OH (US); JASON L. HARRIS, LEBANON, OH (US)

(21) Appl. No.: 18/484,807

(22) Filed: Oct. 11, 2023

Related U.S. Application Data
(60) Provisional application No. 63/522,660, filed on Jun. 22, 2023.

(51) Int. Cl. A61B 17/072; A61L 31/06; A61L 31/10; A61L 31/12; A61L 31/14

(52) U.S. Cl. CPC A61B 17/07292; A61L 31/06; A61L 31/10; A61L 31/129; A61L 31/146; A61B 2017/00526; A61L 2420/02

(57) ABSTRACT

The disclosed technology includes a surgical adjunct that includes a polyurethane foam. A volumetric ratio of the polyurethane foam to the total volume of the surgical adjunct may be in a range of about 0.125 to about 0.325. A glass transition temperature of the surgical adjunct may be about 0° C. to about 40° C.

### CROSS-REFERENCE TO RELATED APPLICATIONS

This application claims priority under 35 U.S.C. § 119 to U.S. Provisional Patent Application No. 63/522,660, filed Jun. 22, 2023, the entire contents of which are fully incorporated herein by reference.

### FIELD

The present invention relates generally to compressible surgical adjuncts, cartridges, cartridge assemblies and methods of making adjuncts and cartridge assemblies.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and is often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

### SUMMARY

There is provided, in accordance with an example of the present invention, a surgical adjunct that includes a polyurethane material. A volumetric ratio of the polyurethane material to the total volume of the surgical adjunct is in a range of about 0.125 to about 0.325. A glass transition temperature of the surgical adjunct is about 0° C. to about 40° C.

There is provided, in accordance with an example of the present invention, a method of making a surgical adjunct. The method includes selectively adding a plasticizer to a polyurethane material to generate the surgical adjunct. The surgical adjunct has a glass transition temperature of about 0° C. to about 40° C.

There is provided, in accordance with an example of the present invention, a surgical adjunct that includes a polyurethane material. A glass transition temperature of the surgical adjunct 604 is about 0° C. to about 40° C.

There is provided, in accordance with an example of the present invention, a surgical staple cartridge assembly, including a cartridge having a length of about 80 mm to about 90 mm and a width of about 8.9 mm to about 14 mm. The surgical staple cartridge assembly also includes a surgical adjunct disposed on the cartridge. The surgical adjunct includes a polyurethane material comprising a volumetric ratio of the polyurethane material to the total volume of the surgical adjunct 604 is in a range of about 0.125 to about 0.325 and a plasticizer added to the polyurethane material. A glass transition T_{g} temperature of the surgical adjunct is about 0° C. to about 40° C. The surgical adjunct having a length of about 40 mm to about 80 mm, a width of about 8 mm to about 12 mm; and a height of about 2.5 mm to about 3.5 mm.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument.

FIG. 2A is a top view of a staple cartridge for use with the surgical stapling and severing instrument of FIG. 1;

FIG. 2B is a side view of the staple cartridge of FIG. 2A;

FIG. 3 is a side view of a staple in an unfired (pre-deployed) configuration that can be disposed within the staple cartridge of the surgical cartridge assembly of FIG. 2A;

FIG. 4 is a perspective view of a knife and firing bar (“E-beam”) of the surgical stapling and severing instrument of FIG. 1;

FIG. 5 is a perspective view of a wedge sled of a staple cartridge of the surgical stapling and severing instrument of FIG. 1;

FIG. 6A is a longitudinal cross-sectional view of an exemplary surgical cartridge assembly having a compressible non-fibrous adjunct attached to a top or deck surface of a staple cartridge;

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct;

FIG. 6C is a perspective view of an exemplary surgical end effector having a channel and a surgical cartridge with a sled and drivers;

FIG. 6D is a perspective view of an exemplary sled for an exemplary surgical cartridge;

FIG. 6E is a perspective view of an exemplary single driver for an exemplary surgical cartridge;

FIG. 6F is a front view of the exemplary single driver of FIG. 6E;

FIG. 6G is a perspective view of an exemplary double driver for an exemplary surgical cartridge;

FIG. 6H is a front view of an exemplary double driver of FIG. 6G;

FIG. 6I is a cutaway view of an exemplary cartridge in an exemplary end effector;

FIG. 7 is a partial-schematic illustrating the adjunct of FIGS. 6A-6B in a tissue deployed condition;

FIG. 8A is a perspective view of an exemplary cartridge assembly;

FIG. 8B is a side view of an exemplary adjunct for a cartridge assembly;

FIG. 8C is a top view of an exemplary adjunct for a cartridge assembly;

FIG. 8D is a front view of an exemplary adjunct for a cartridge assembly;

FIG. 8E is a diagram showing an enlarged portion of an exemplary adjunct with a porous structure;

FIG. 9A is a side view of an exemplary adjunct compressed in a dog bone profile;

FIG. 9B is a front view of FIG. 9A.

FIG. 10A is a perspective view of an exemplary cartridge deck with three levels;

FIG. 10B a perspective view of an enlarged front portion of exemplary cartridge deck of FIG. 10A;

FIG. 10C a perspective view of an enlarged rear portion of exemplary cartridge deck of FIG. 10A;

FIG. 10D a top view of an enlarged front portion of exemplary cartridge deck of FIG. 10A;

FIG. 11A is a perspective view of an exemplary cartridge deck with two levels;

FIG. 11B a perspective view of an enlarged front portion of exemplary cartridge deck of FIG. 11A;

FIG. 11C a perspective view of an enlarged rear portion of exemplary cartridge deck of FIG. 11A;

FIG. 11D a top view of an enlarged front portion of exemplary cartridge deck of FIG. 10A;

FIG. 12A is a top perspective view of an exemplary adjunct after use;

FIG. 12B is a side view of an exemplary adjunct after use;

FIG. 13 is graph showing the glass transition temperature T_{g} of an exemplary adjunct; and

FIG. 14 is a flow chart showing an exemplary method for making an adjunct.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 90%” may refer to the range of values from 81% to 99%.

Surgical stapling assemblies and methods for manufacturing and using the same are provided. In general, a surgical stapling assembly can include a staple cartridge having staples disposed therein and an adjunct configured to be releasably retained on the staple cartridge. As discussed herein, the various adjuncts provided can be configured to compensate for variations in tissue properties, such as variations in tissue thickness, and/or to promote tissue ingrowth when the adjuncts are stapled to tissue.

An exemplary stapling assembly can include a variety of features to facilitate application of a surgical staple, as described herein and illustrated in the drawings. However, a person skilled in the art will appreciate that the stapling assembly can include only some of these features and/or it can include a variety of other features known in the art. The stapling assemblies described herein are merely intended to represent certain exemplary examples. Moreover, while the adjuncts are described in connection with surgical staple cartridge assemblies, the adjuncts can be used in connection with staple reloads that are not cartridge based or any type of surgical instrument.

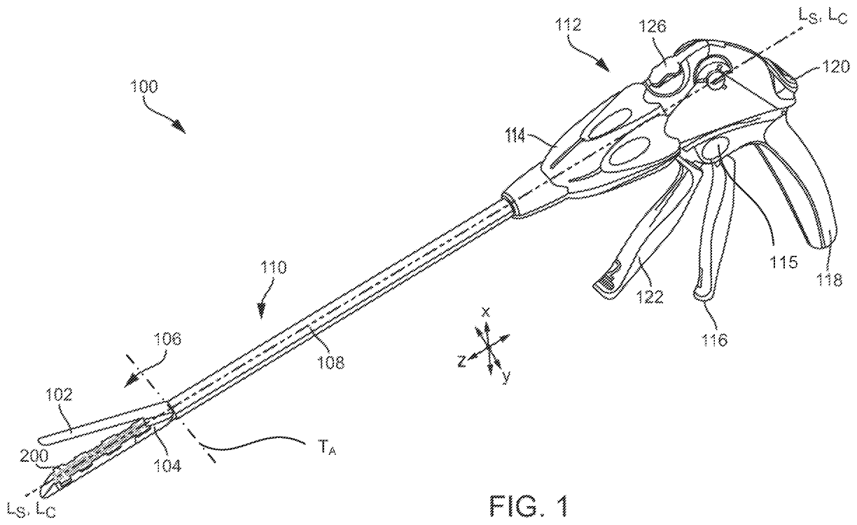

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the staple applying assembly 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108 forming an implement portion 110. When the end effector 106 is closed, or at least substantially closed, (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (T_{A}) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be a powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

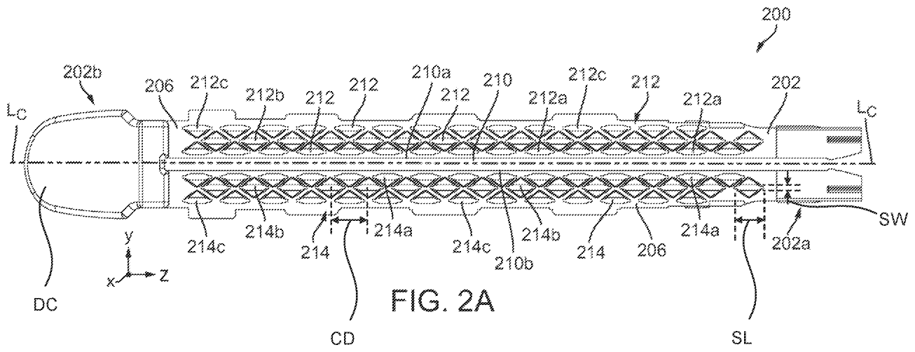

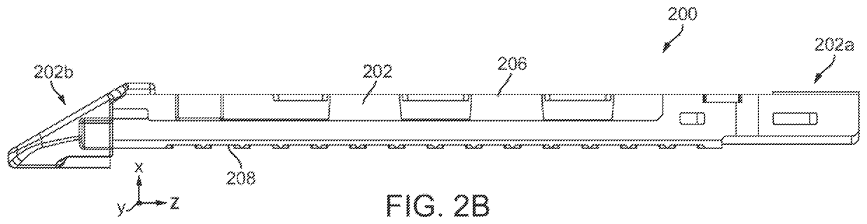

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B, has a proximal end 202a and a distal end 202b with a cartridge longitudinal axis (LC) extending therebetween. As a result, when the staple cartridge 200 is inserted into the elongate channel 104 (FIG. 1), the longitudinal axis (LC) is substantially or approximately parallel with the longitudinal axis (LS) of the elongate shaft 108. Further, the staple cartridge 200 includes a longitudinal slot 210 defined by two opposing walls 210a, 210b and configured to receive at least a portion of a firing member of a firing assembly, like firing assembly 400 in FIG. 4, as discussed further below. As shown, the longitudinal slot 210 extends from the proximal end 202a toward the distal end 202b of the staple cartridge 200. It is also contemplated herein that in other examples, the longitudinal slot 210 can be omitted.

The illustrated staple cartridge 200 includes staple cavities 212, 214 defined therein, in which each staple cavity 212, 214 is configured to removably house at least a portion of a staple (not shown). The number, shape, and position of the staple cavities can vary and can depend at least on the size and shape (e.g., mouth-like shape) of the staples to be removably disposed therein. In this illustrated example, the staple cavities are arranged in two sets of three longitudinal rows, in which the first set of staple cavities 212 is positioned on a first side of the longitudinal slot 210 and the second set of staple cavities 214 is positioned on a second side of the longitudinal slot 210. On each side of the longitudinal slot 210, and thus for each set of rows, a first longitudinal row of staple cavities 212a, 214a extends alongside the longitudinal slot 210, a second row of staple cavities 212b, 214b extends alongside the first row of staple cavities 212a, 214a, and a third row of staple cavities 212c, 214c extends alongside the second row of staple cavities 212b, 214b. Each row may be approximately parallel and the staple cavities that make up the rows may be approximately parallel in orientation with the longitudinal slot 210. As shown in FIGS. 2A, each staple cavity 212, 214 may include a maximum length SL of about 0.122 inches to about 0.124 inches and a maximum width SW of about 0.023 inches to about 0.027 inches. In addition, at least the centers of two adjacent cavities 212, 214 are spaced apart by about 0.158 inches.

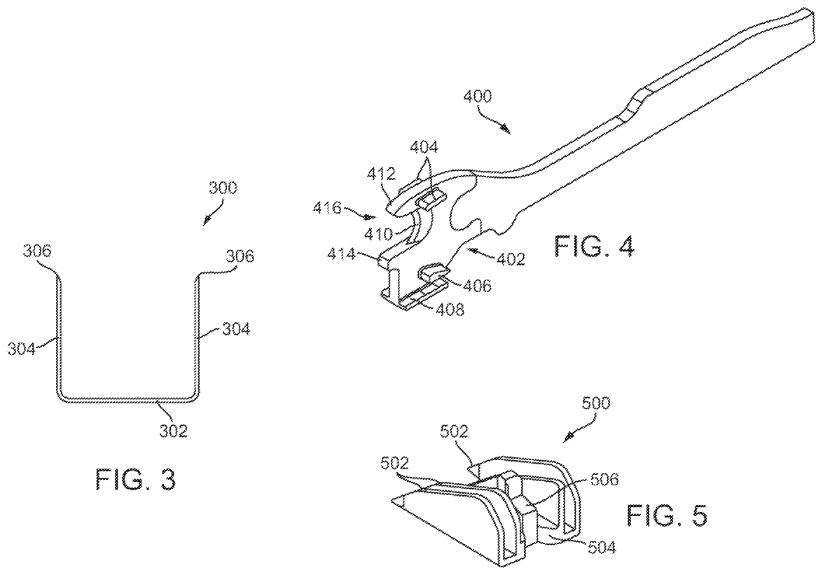

The staples releasably stored in the staple cavities 212, 214 can have a variety of configurations. An exemplary staple 300 that can be releasably stored in each of the staple cavities 212, 214 is illustrated in FIG. 3 in its unfired (pre-deployed, unformed) configuration. The illustrated staple 300 includes a crown (base) 302 and two legs 304 extending from each end of the crown 302. In this example, the crown 302 extends in a linear direction and the staple legs 304 have the same unformed height. Further, prior to the staples 300 being deployed, the staple crowns 302 can be supported by staple drivers that are positioned within the staple cartridge 200 and, concurrently, the staple legs 304 can be at least partially contained within the staple cavities 212, 214. Further, the staple legs 304 can extend beyond a top surface, like top surface 206, of the staple cartridge 200 when the staples 300 are in their unfired positions. In certain instances, as shown in FIG. 3, the tips 306 of the staple legs 304 can be pointed and sharp which can incise and penetrate tissue.

In use, staples 300 can be deformed from an unfired position into a fired position such that the staple legs 304 move through the staple cavities 212, 214, penetrate tissue positioned between the anvil 102 and the staple cartridge 200, and contact the anvil 102. As the staple legs 304 are deformed against the anvil 102, the legs 304 of each staple 300 can capture a portion of the tissue within each staple 300 and apply a compressive force to the tissue. Further, the legs 304 of each staple 300 can be deformed downwardly toward the crown 302 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed, unformed) configuration. In other examples, the staples can include at least two groups of staples each having a different unfired (pre-deployed, unformed) configuration, e.g., varying in height and/or shape, relative to one another, etc.

Referring back to FIGS. 2A-2B, the staple cartridge 200 extends from a top surface or deck surface 206 to a bottom surface 208, in which the top surface 206 is configured as a tissue-facing surface and the bottom surface 208 is configured as a channel-facing surface. As a result, when the staple cartridge 200 is inserted into the elongate channel 104, as shown in FIG. 1, the top surface 206 faces the anvil 102 and the bottom surface 208 (obstructed) faces the elongate channel 104.

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that effects staple formation by the end effector 106.

In use, the anvil 102 in FIG. 1 can be moved into a closed position by depressing the closure trigger in FIG. 1 to advance the E-beam 402 in FIG. 4. The anvil 102 can position tissue against at least the top surface 206 of the staple cartridge 200 in FIGS. 2A-2B. Once the anvil has been suitably positioned, the staples 300 in FIG. 3 disposed within the staple cartridge can be deployed.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be essentially fully absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

### Methods of Stapling Tissue

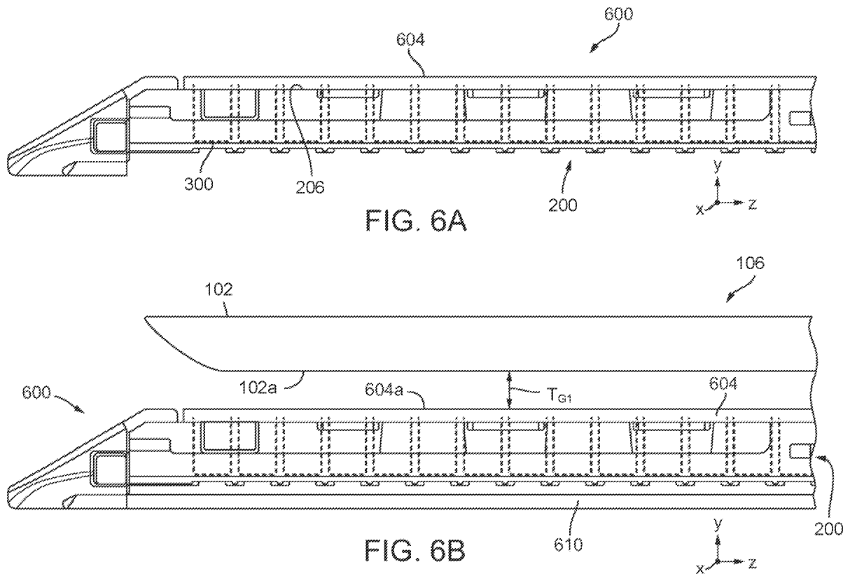

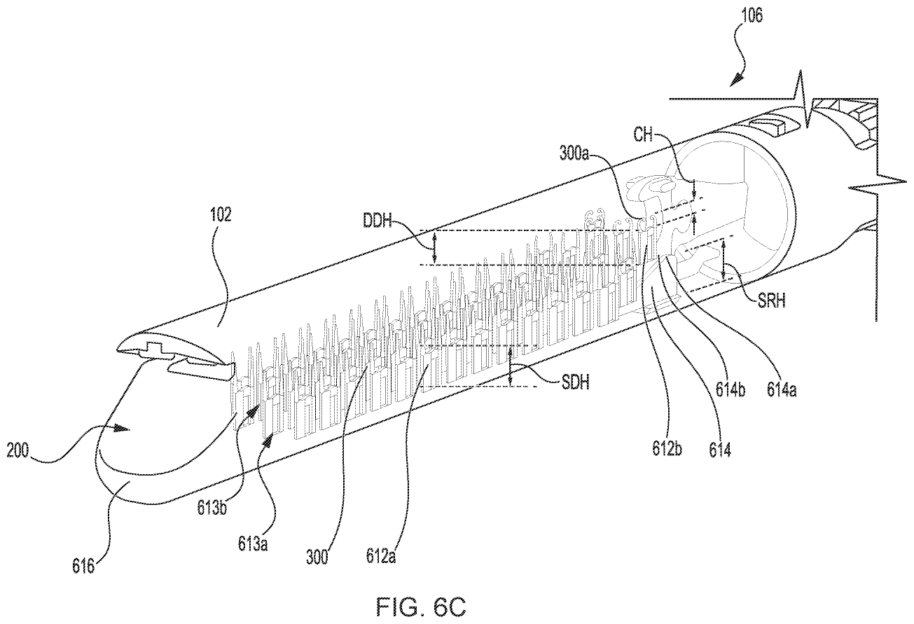

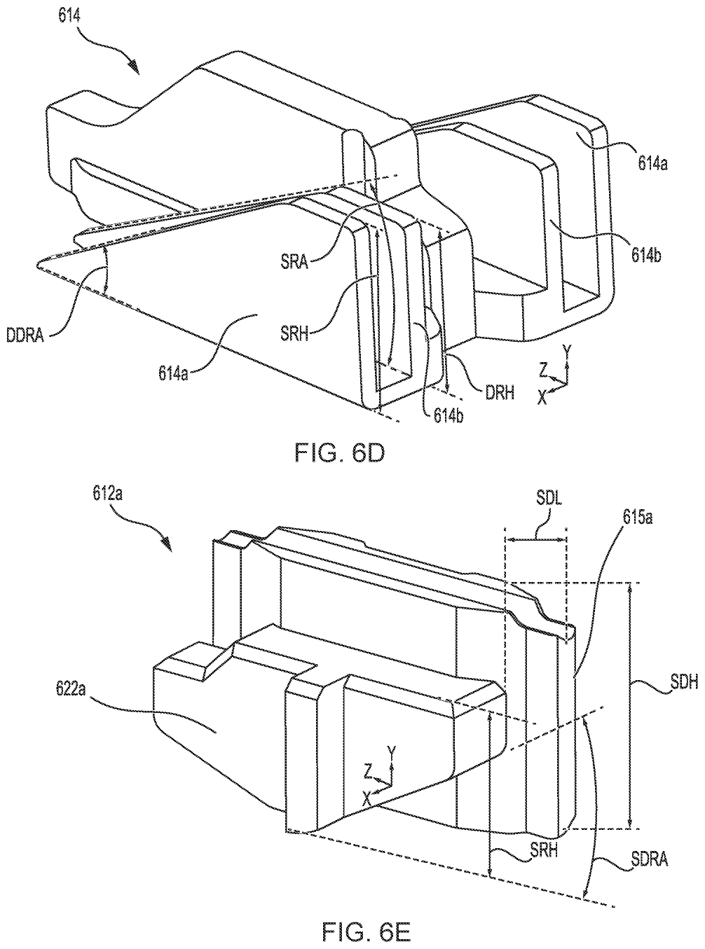

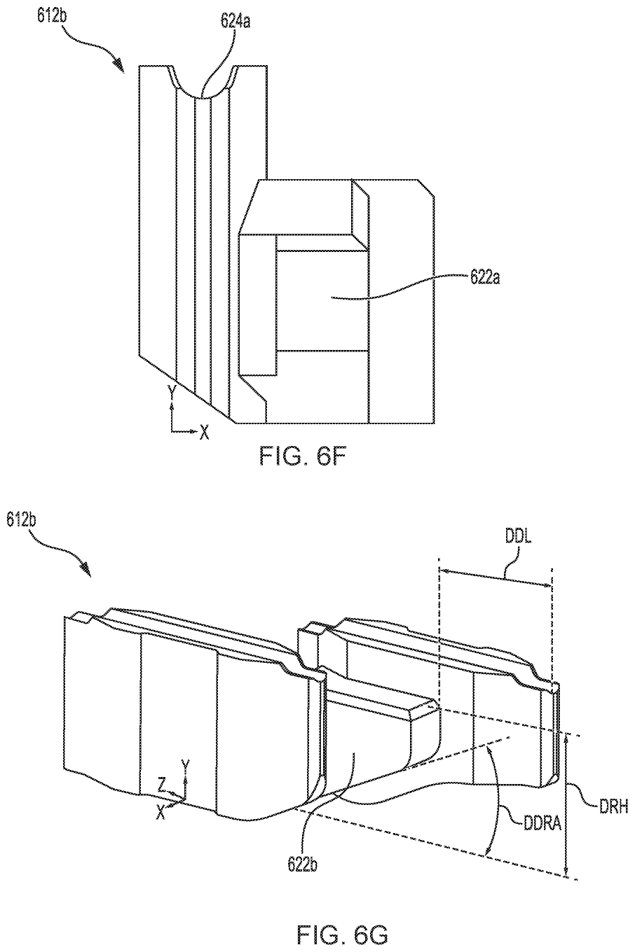

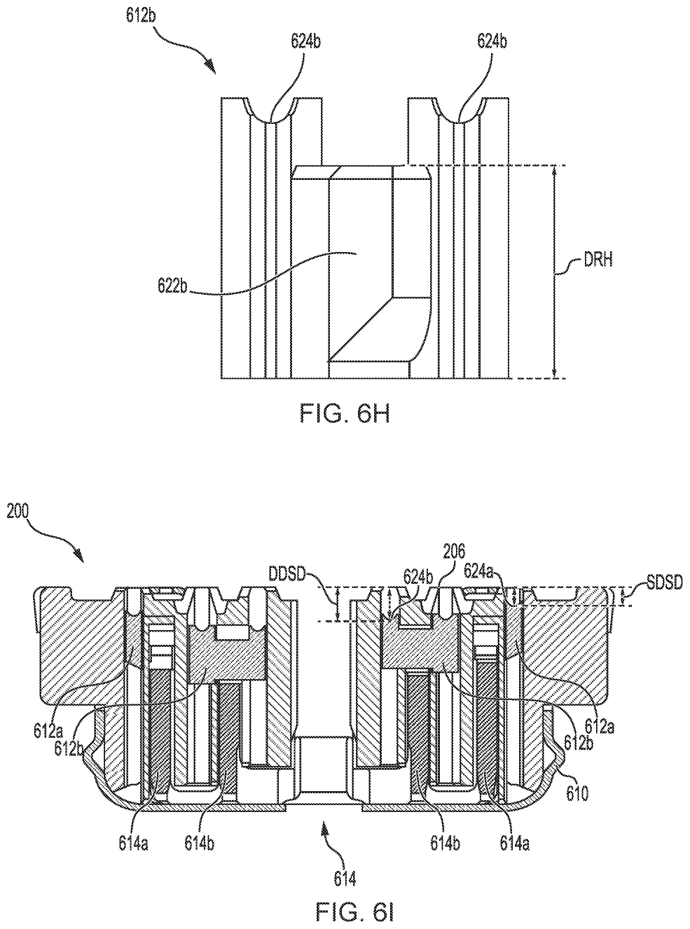

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300, that are configured to be deployed into tissue. The staples 300 can have any suitable unformed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Patent Publication No. 2016/0106427, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap T_{G1} created between the staple cartridge 602 and the anvil 612. More specifically, the tissue gap T_{G1} is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap T_{G1} is generally uniform (e.g., nominally identical within manufacturing tolerances) when no tissue is disposed therein. In other words, the tissue gap T_{G1} is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap T_{G1} can be varied.

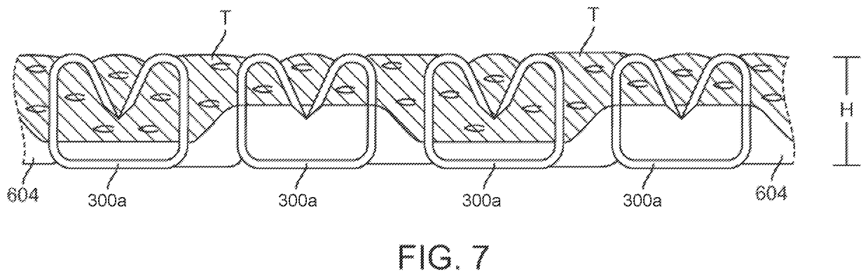

The adjunct 604 is compressible to permit the adjunct to compress to varying heights to thereby compensate for different tissue thickness that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7). That is, the adjunct 604 can have an undeformed state in which a maximum height of the adjunct 604 is greater than a maximum height of a fired staple (e.g., a staple that is in a formed configuration).

As shown in FIG. 6C, the staple cartridge 200 includes a sled 614 and a plurality of drivers 612 configured to drive one or more staples in an upward direction to deploy the staples when a user presses a firing trigger 122 shown in FIG. 1. Once the firing trigger 122 is pressed, the sled 614 moves toward the distal end 616 of the end effector 106 contacting one or more drivers 612a, 612b at a time forcing the one or more drivers 612a, 612b upward along with one or more corresponding staples 300 upward to form fired staple 300a and to capture a material, such as tissue (T), (see FIG. 7.) between the anvil 104 and the fired staple 300a. The cartridge 200 may include a first row 613a of single drivers 612a that corresponds to driving staples 300 positioned in the third row of staple cavities 212c, 214c and a second row 613b of double drivers 612b that corresponds to driving staples 300 positioned in a first row of staple cavities 212a, 214b and a third row 212a, 212b (see FIG. 2A for staple cavities).

Referring to FIGS. 6C-6I, one or more single drivers 612a may have a height SDH of about 0.044 inches to about 0.074 inches, such as about 0.050 inches to about 0.068 inches, about 0.054 inches, or about 0.06 inches. One or more double drivers 612b may have a height DDH of about 0.044 inches to about 0.074 inches, such as about 0.050 inches to about 0.068 inches, about 0.054 inches, or about 0.06 inches. The sled 614 may have a least a first rail 614a corresponding to the single drivers 612a positioned in the first row 613a and a second rail 614b corresponding to the double drivers 612b positioned in the second row 613b. The first rail 614a may have a rail height SRH of about 0.164 inches or about 0.167 inches and engages with the single driver 612a. The second rail 614b may have a rail height DRH of about 0.140 inches to about 0.162 inches such as about 0.149 inches or about 0.152 inches and engages with the double driver 612b. Once the staples 300 are deployed, they form a fired staple 300a with a crush height CH of about 0.08 inches to about 0.12 inches such as about 0.97 inches or about 0.1 inches.

Referring to FIG. 6I, the saddles 624a of single drivers 612a may be spaced apart from the cartridge deck 206 a distance SDSD when the single driver 612a is fully engaged with the sled 614 of about −0.010 inches to about −0.031 inches such as −0.021 inches or about −0.017 inches. The saddles 624b of the double driver 612b may be spaced apart from the cartridge deck 206 a distance DDSD when the double driver 612b is fully engaged with the sled 614 about −0.022 inches to about −0.042 inches such as about −0.032 inches or about −0.036 inches.

Referring to FIGS. 6D-6I, one or more single drivers 612a may have a rigger 622a that may have a ramp location SDL that extends about 0.056 inches to about 0.066 inches, such as about 0.061 inches, from a proximal edge 615a of the single driver 612a. Rigger 622a may have a height SRH of about 0.035 inches to about 0.055 inches such as about 0.45 inches or about 0.43 inches. One or more double drivers 612b may have a rigger 622b that may have a ramp location DDL that extends 0.094 inches to about 0.114 inches, such as about 0.104 inches, from the proximal edge 615b of the double driver 612b. Rigger 622b may have a height of about 0.039 inches to about 0.060 inches such as about 0.050 inches or about 0.048 inches. The single driver 612a, may have a ramp angle SDRA of about 24 degrees to about 34 degrees, such as about 29 degrees. The double driver 612b may have a ramp angle DDRA of about 22 degrees to about 30 degrees, such as about 26 degrees. The sled's 614 first rail 614a may have a ramp angle SRA of about 22 to about 32 degrees, such as about 26.9 degrees. The sled's 614 second rail 614b may have a ramp angle SRA of about of about 20 degrees to about 30 degrees, such as about 24.1 to about 24.5 degrees.

In use, once the surgical stapling and severing device, like device 100 in FIG. 1, is directed to the surgical site, tissue is positioned between the anvil 102 and the stapling assembly 600 such that the anvil 102 is positioned adjacent to a first side of the tissue and the stapling assembly 600 is positioned adjacent to a second side of the tissue (e.g., the tissue can be positioned against the tissue-contacting surface 604a of the adjunct 604). Once tissue is positioned between the anvil 102 and the stapling assembly 600, the surgical stapler can be actuated, e.g., as discussed above, to thereby clamp the tissue between the anvil 102 and the stapling assembly 600 (e.g., between the tissue-compression surface 102a of the anvil 102 and the tissue-contacting surface 604a of the adjunct 604) and to deploy staples from the cartridge through the adjunct and into the tissue to staple and attach the adjunct to the tissue.

As shown in FIG. 7, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

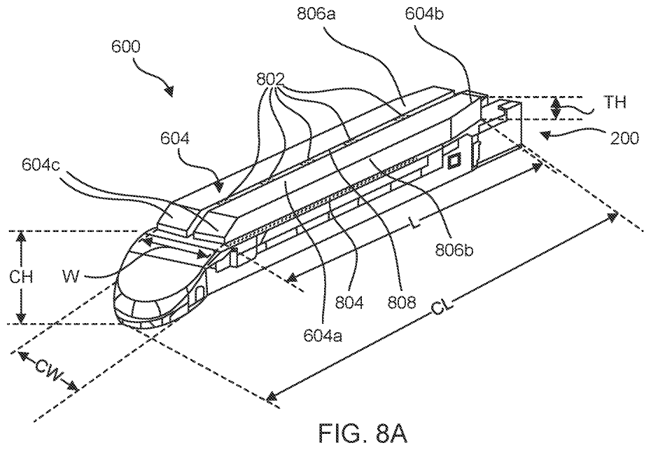

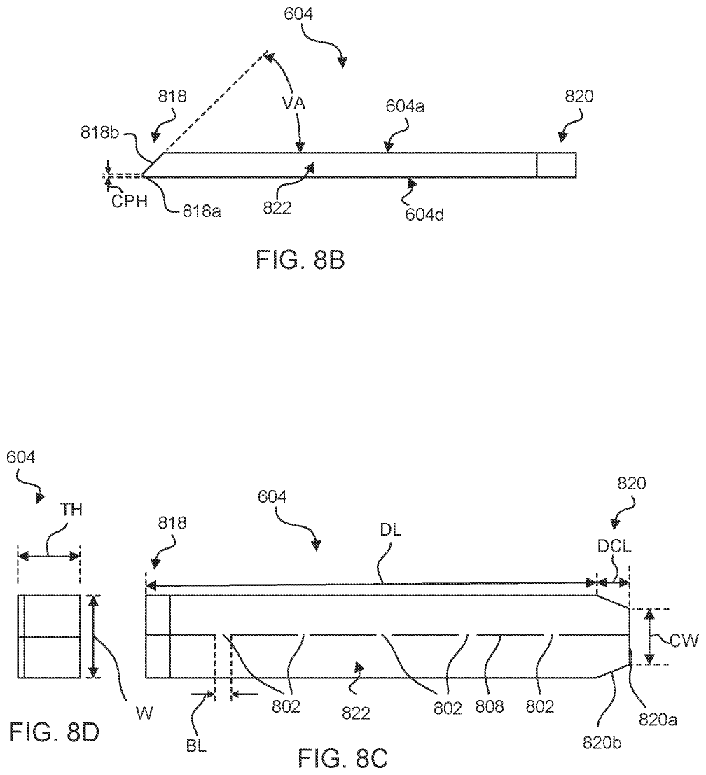

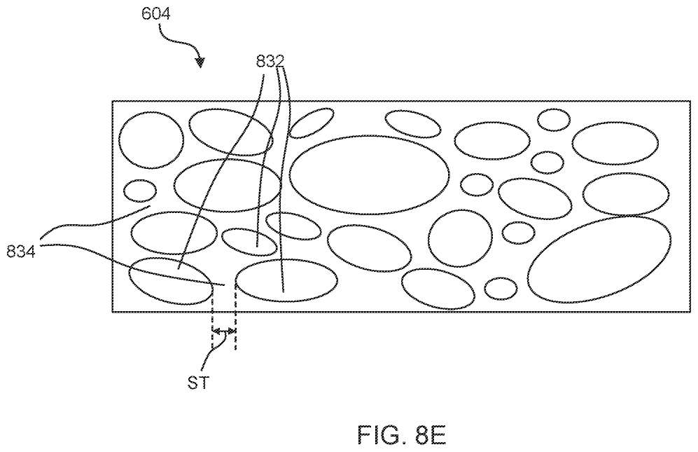

FIG. 8A illustrates a perspective view of a staple cartridge assembly 600 with an adjunct 604 and a staple cartridge 200. The adjunct 604 has a tissue contacting surface 604a, a proximal end 604a, and a distal end 604b. The adjunct 604 may include a slot/slit 808 separating or partially separating two parallel portions of the adjunct 604. In one example, adjunct 604 may include a slot 808 separating two parallel portions of the adjunct 604, while in another example, adjunct 604 may include a slit 808 separating two parallel portions of the adjunct 604 and also one or more bridges (e.g., five bridges) 802 connecting the two parallel portions of the adjunct 604. At least one bridge has a length in the longitudinal direction of about 0.035 inches to about 0.046 inches. The adjunct 604 has a length L of about 40 mm to about 80 mm, such as about 60 mm to about 65 mm, about 66.04 mm to about 66.3 mm, about 45 mm to about 55 mm, or about 51.12 mm to about 51.38 mm. The adjunct 604 has a width W of about 8 mm to about 12 mm, such as about 9.75 mm to about 10.25 mm or about 10.025 mm to about 10.035 mm. The adjunct 604 may also have a thickness or height TH of about 2.5 mm to about 3.5 mm, such as about 2.85 mm to about 3.15 mm or about 2.95 mm to about 3.05 mm.

The cartridge 200 has a height CH of about 6.3 mm to about 8.1 mm, a width CW of about 8.9 mm to about 14 mm, and a length CL of about 80 to about 90 mm such as about 86.7 mm.

Referring to FIGS. 8B-8D, the adjunct 604 has a lower surface 604d and may have a distal chamfered portion 818, a proximal chamfered portion 820, and a center portion 822. The distal chamfered portion 818 has a vertical portion 818a having a height CPH of about 0.009 inches to about 0.029 inches, such as about 0.019 inches. The distal chamfered portion 818 may have an angled portion 818b proximal the vertical portion 818a. The angled portion 818b has a slope VA of about 30 degrees to about 60 degrees, such as about 45 degrees, measured from the tissue contacting surface 604a.

Referring to FIG. 8C, the distal chamfered portion 818 and the center portion 822 has a combined length DL of about 2.25 inches to about 2.45 inches, such as about 2.35 inches. The proximal chamfered portion 820 has an angled portion 820b with a length DCL of about 0.1 inches to about 0.3 inches. In addition, the proximal chamfered portion 820 has a horizontal portion 820a and an angled portion 820n. The horizontal portion 820a may have a width CW of about 0.27 inches to about 0.29 inches, such as about 0.28 inches.

In some examples, the adjunct 604 includes one or more slits 808 with two or more bridges 802 spaced apart by a bridge length BL of about 0.035 inches to about 0.045 inches such as about 0.04 inches.

Referring back to FIG. 8A, the staple cartridge 200 may include one or more raised ledges 804 along one or more sides of the adjunct 602 to help align the adjunct 604 on the deck of the staple cartridge 200.

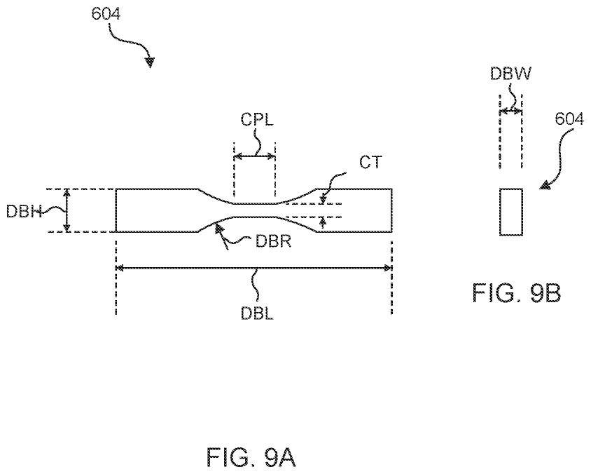

As previously mentioned, the adjunct 604 is compressible. FIGS. 9A and 9B show the dog-bone shape and dimensions that the adjunct 604 takes when subjected to compressibility tests to determine its material properties. As shown, the adjunct 604 has a compression length CPL of about 9.45 mm to about 9.61 mm, such as about 9.53 mm. The adjunct 604 has a compression thickness CT of about 3.15 mm to about 3.21 mm, such as about 3.18 mm. The adjunct 604 has dog bone radius DBR of about 12.62 mm to about 12.78 mm, such as about 12.7 degrees. The adjunct 604 has a dog bone height DBH of about 6.35 mm to about 12.71 mm, such as about 9.53 mm. The adjunct 604 has a dog bone length DBL of about 40 mm to about 80 mm, such as about 63.5 mm. The adjunct has a dog-bone width DBW of about 2.5 mm to about 3.5 mm, such as about 3.03 mm.

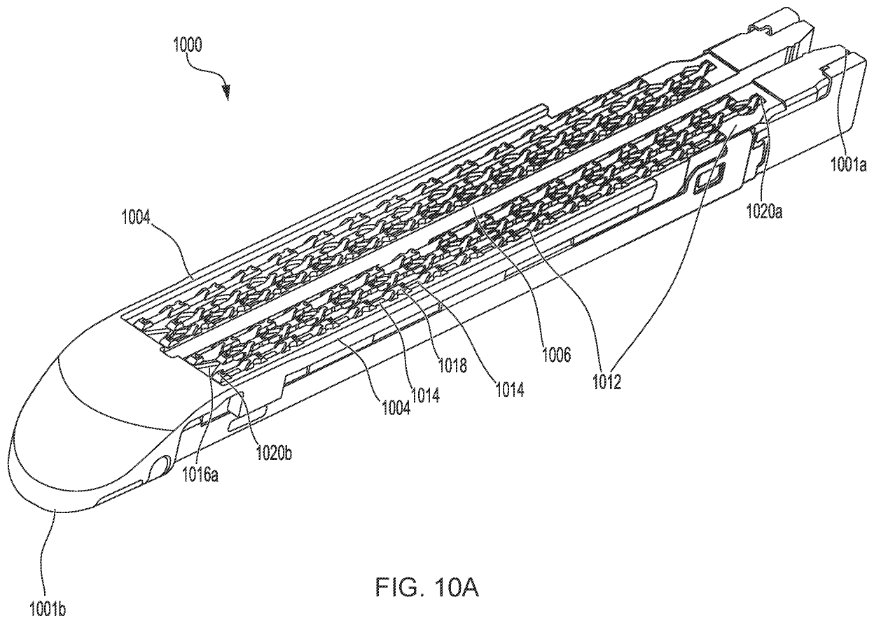

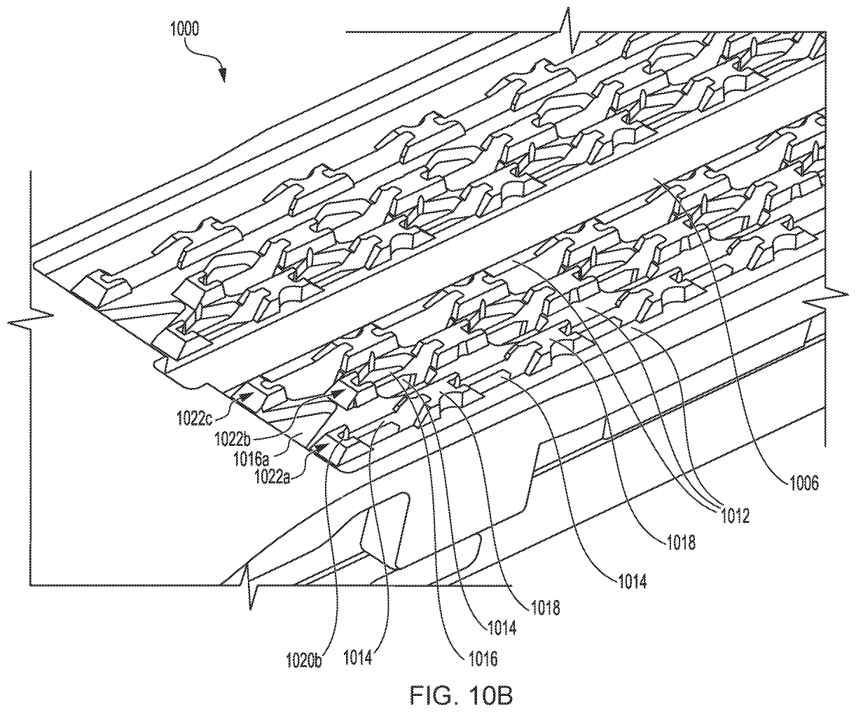

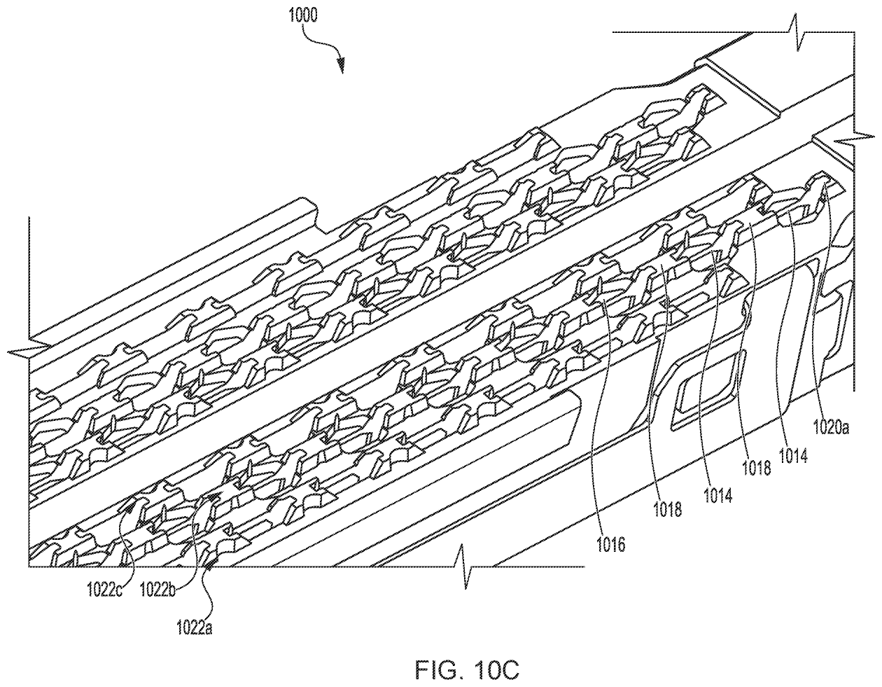

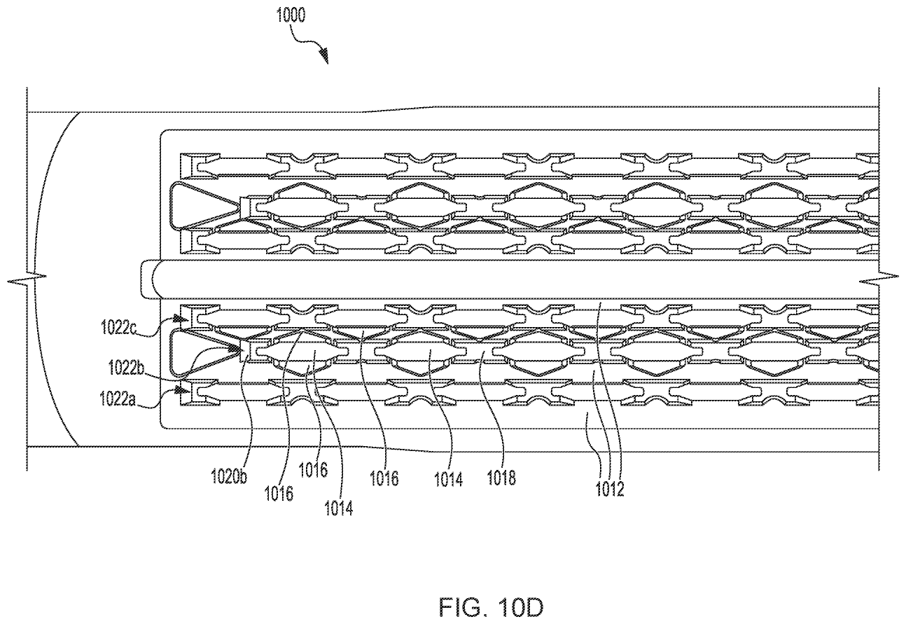

FIGS. 10A-10D illustrate an exemplary staple cartridge 1000 with a deck 1012 having three levels so that the adjunct 604 may expand around the various protrusions and recessions to increase the contact surface area between the adjunct 604 and the staple cartridge 1000 to increase the adherence and alignment between the staple cartridge 1000 the adjunct 604. Staple cartridge 1000 is similar to staple cartridge 200, but with more exemplary details showing the deck 1012. In particular, the staple cartridge 1000 may include a distal end 1001b having an atraumatic shape and a proximal end 1001a. The staple cartridge 1000 may include one or more raised ledges 1004 (such as two raised ledges 104) aligning with longitudinal edges of the deck 1012. The staple cartridge 1000 may include a plurality of staple cavities 1014 positioned and aligned in a first row 1022a, a second row 1022b, and a third row 100c on each side of cartridge slot 1006. In each row, at least two adjacent staple cavities 1014 are separated by a raised surface 1018 or combined raised surface of the deck 1012 that conforms to the ends of the staple cavities 1014. This combined raised surface 1018 essential combines a raise surface corresponding proximal end of an adjacent staple cavity 1014 with a distal end of a staple cavity 1014. In addition, staple cavities 1014 positioned at the distal end 1001b and the proximal end 1001a of the first row 1022a, the second row 1022b, and the third row 100c include raised surfaces 1020b, 1020a that are approximately half the size of the combined raised surfaces 1018 between staple cavities 1014. The cartridge deck 1012 may also include a plurality of lowered surfaces 1016. At least two triangular shaped lower surfaces 1016 may be located at the lateral sides of the staples cavities 1014 located in the second row 1022b with an apex of the triangle pointing toward combined raised surfaces 1018 located in adjacent rows 1022a, 1022c of staple cavities 1014. At least one triangular shaped lower surface 1018 located at a lateral side of the third row 1022c distal to the slot 1006 may have an apex of the triangle pointing toward combined raised surface 1018 located in adjacent row 1022b. In addition, staple cartridge 1000 may include a triangular-shaped end lower surface 1016a positioned between a distal end 1001b of the cartridge 1000 and the second row 1022b of staple cavities 1014 with an apex of the aligned with the second row 1022b and pointing toward to the proximal end 1001a of the staple cartridge 1000. As shown, cartridge 1000 may not include any lowered surfaces directly adjacent to staple cavities 1014 of the first row 1022a.

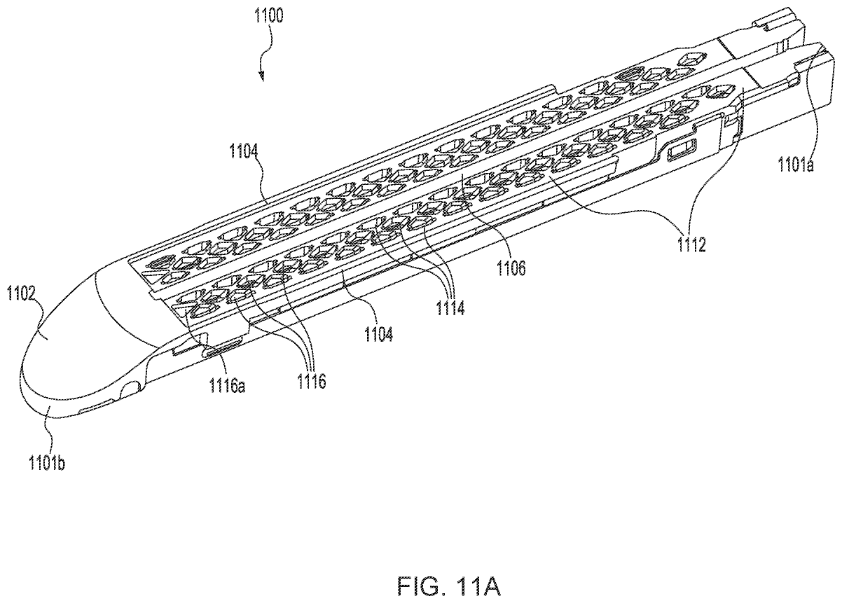

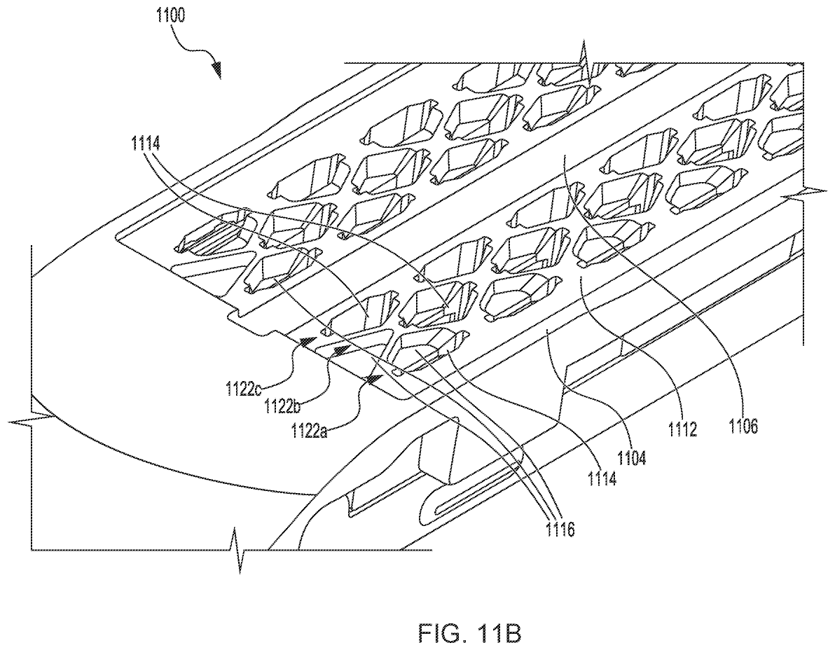

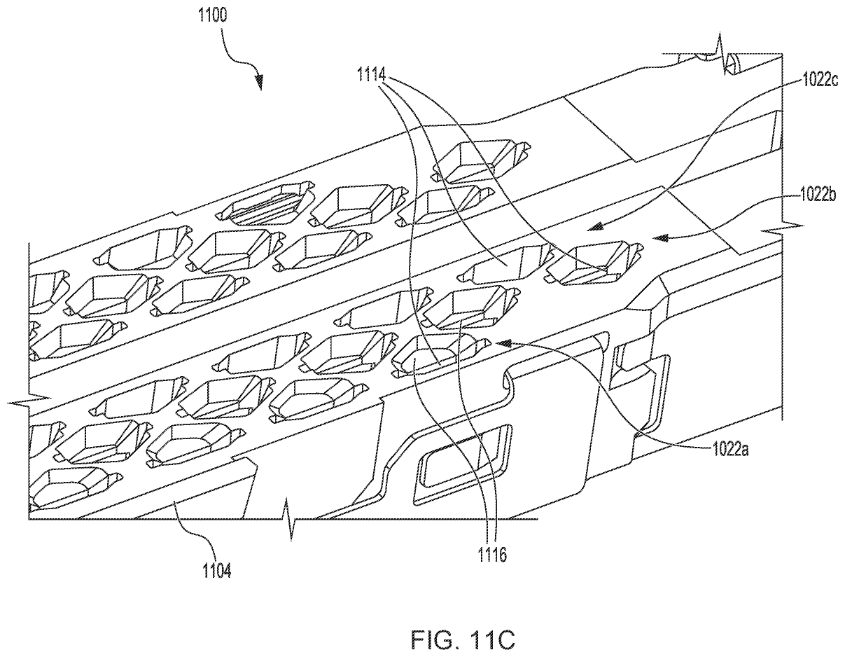

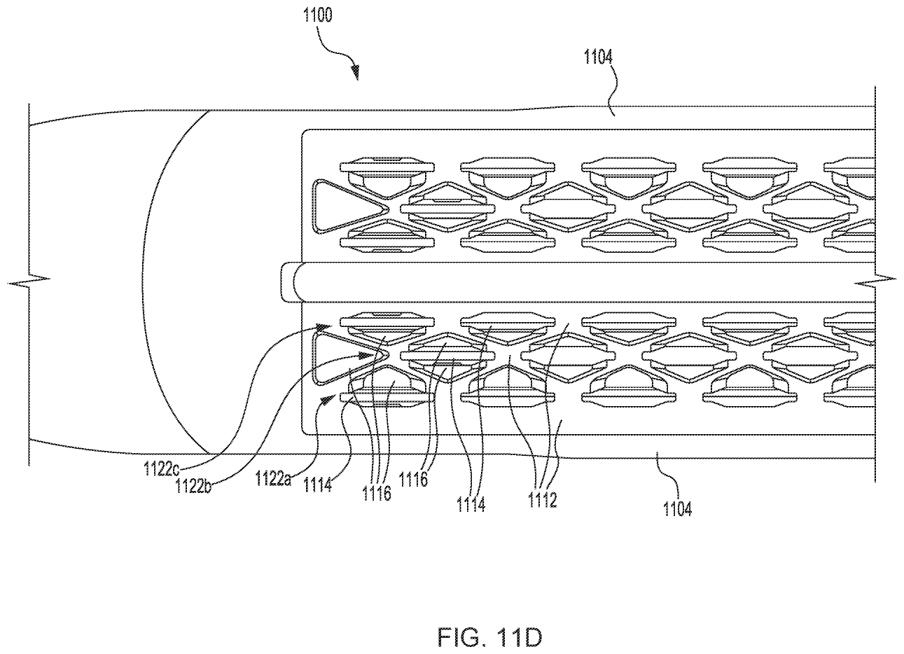

FIGS. 11A-11D illustrate an exemplary staple cartridge 1100 similar to exemplary staple cartridge 1000 of FIGS. 10A-10D, with a few notable differences such as having a flat deck 1112 without raised surfaces adjacent staple cavities 1114. Deck 1112 may include a plurality of lowered surfaces 1116, 1116a positioned and shaped similarly to lowered surfaces 1016, 1016a of exemplary cartridge 1000. In addition, deck 1112 may include additional lowered surfaces 1116 positioned adjacent staple cavities 1114 of the first row 1122a on the side closest to the slot 1106. Thus, each staple cavity 1114 of the first, second, and third rows 1122a, 1122b, 1122c may have at least on adjacent lower surface 1016 that is triangular-shaped.

Both staple cartridges 1000 and 1100 allow for staples 300 having a pre-formed height about 0.179 inches, which is more than previous devices, with a deck height of about 0.060 inches, which is shorter than previous devices.

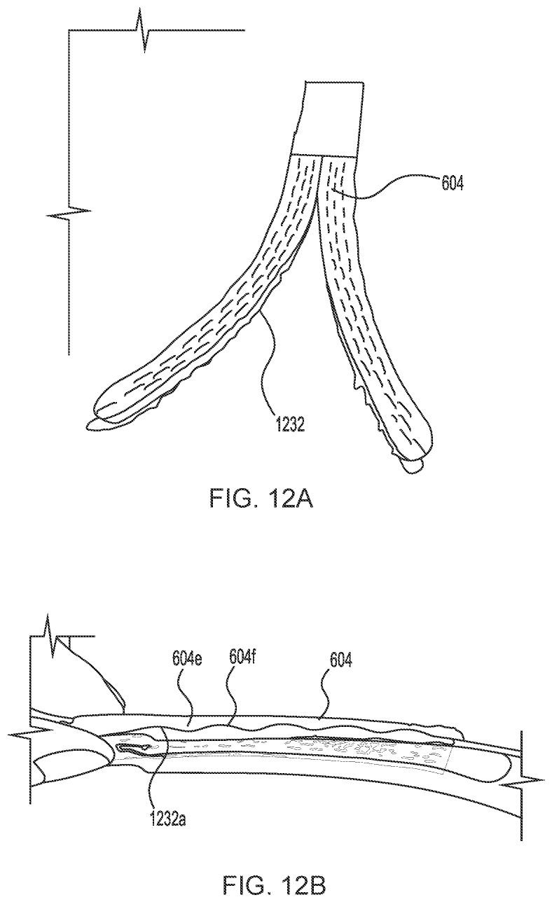

FIGS. 12A and 12B show top and side view of an adjunct 604 after firing staple. As can be seen, adjunct 604 may split in two post firing and the adhesive 1232 adhering the adjunct 604 to the cartridge 200 (see FIGS. 6A and 6B) may with the adjunct 604 form bumps 604e, 604f in the adjunct and corresponding bumps 1232a. This means that adjunct 604 is able to adapt to different heights and compressions depending on the application. An adjunct may be attached to the cartridge 200 with about 100 mg to about 120 mg of the adhesive or buttress adhesive material.

The surgical adjunct 604 may have one or more of the properties described below to enable the adjunct to be flexible when in vivo, but yet remain in a certain position attached to the cartridge when outside of the body. For example, the polyurethane may server to create an adjunct 604 that is flexible when going into the body but “sets” to its final mechanical properties as the plasticizer is absorbed in vivo. In some examples, a plasticizer may be added to the polyurethane foam to lower its glass transition temperature to be within the below described ranges as well as conform to the other listed properties. Regardless, an adjunct 604 having one or more of the below properties consistently creates a hemostatic or near hemostatic seal on tissue.

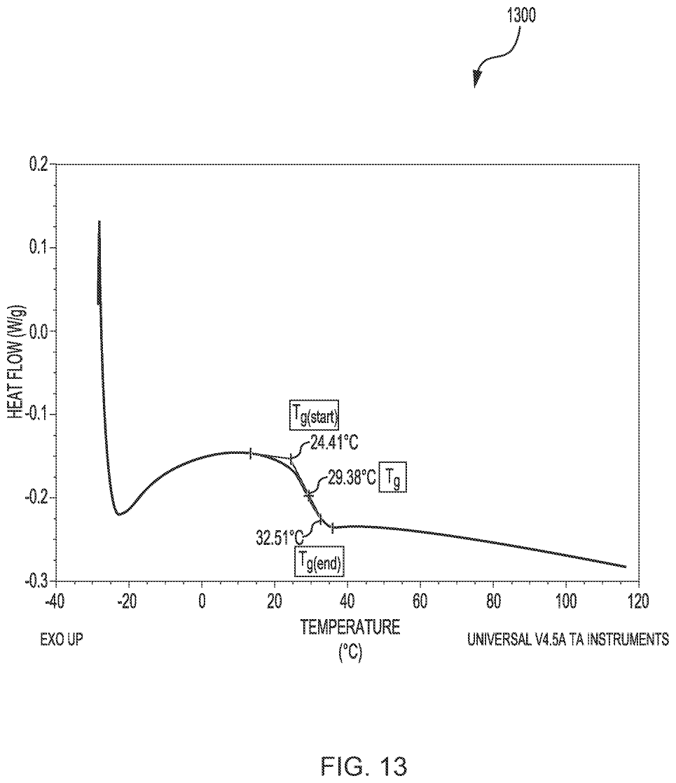

The surgical adjunct 604 may include a polyurethane foam with or without a plasticizer where the glass transition temperature of the surgical adjunct 604 is about 0° C. to about 40° C. (e.g., about 19.4° C.), such as about 7.5° C. to about 22.5° C. or about 12.5° C. to about 17.5° C. The glass transition temperature of the adjunct 604 is obtained by using a standard differential scanning calorimetry (DSC) system. Using the DSC system with its output shown in FIG. 13, an adjunct 604 was equilibrated at about −40° C., heated at about 40° C./min to about 120° C., held isothermally for about 1 minute, cooled at about 40° C./min to about −40° C., held isothermally for about 1 minute, and then heated at about 10° C./min to about 120° C., where the glass transition temperature T_{g} was measured and recorded by the DSC system.

The surgical adjunct 604 may include a volumetric ratio of the polyurethane foam to the total volume of the adjunct 604 of about 0.125 to about 0.325, such as about 0.175 to about 0.225 or about 0.19 to about 0.21. The total volume may include air (from pores of the foam) or other material beside the foam structures.

The plasticizer may include one or more of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid. In some examples, the plasticizer includes one or more fatty acids.

Referring to FIG. 8E, the adjunct 604 may have pores 832 with a median pore size of about 0.025 mm³ to about 0.300 mm³, such as about 0.022 mm³. In some examples, the adjunct 604 may have one or more struts 834 between the pore 832 that provide support and strength to the adjunct 604. In particular, the adjunct 604 may include a plurality of struts 834, having a median strut thickness ST of about 0.025 mm to about 0.300 mm, such as about 0.08 mm.

In some examples, the adjunct 604 includes a polydioxanone (PDO) film disposed on one or more surfaces of the polyurethane foam. In some examples, the PDO film is adhered to at least a bottom or crown side of the adjunct 604. In some examples, the PDO film has a thickness of about 20 μm to about 100 μm, such as about 40 μm.

The adjunct 604 may have a compression strength of about 30 kPa to about 70 kPa, such as about 30 kPa to about 60 kPa (e.g., about 42 kPa), about 30 kPa to about 50 kPa, about 32.5 kPa to about 37.5 kPa. In order to test compression strengths, an adjunct 604 was placed in a humid warm environment at approximately 37° C., compressed to a first height, then a second height shorter than the first height, and then released back to the first height at which point the adjunct's compression strength was measured.

In some examples, the foam (e.g., polyurethane foam) portion of the adjunct 604 may have a (peak) tensile strength of about 50 kPa to about 150 kPa or about 30 kPa to about 90 kPa such as about 45 kPa to about 85 kPa or about 55 kPa to about 75 kPa. In some examples, the foam (e.g., polyurethane foam) of the adjunct 604 will have (peak) tensile strength of about 110 kPa to about 150 kPa. Tensile strength is measured on an adjunct 604 having the dog-bone configuration shown and described with respect to FIGS. 9A and 9B. Specifically, the adjunct's 604 tensile strength is measured after submerging it in water at a temperature of about 37° C. for less than a minute and then running a tensile strength test.

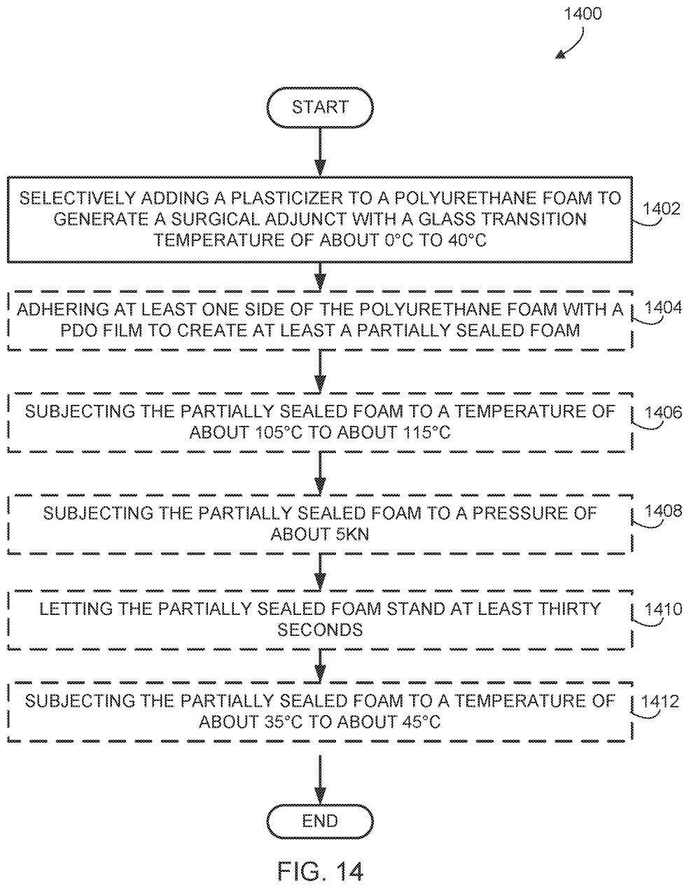

Referring to FIG. 14, a surgical adjunct 604 may be made by using method 1400, which includes selectively (step 1402) adding a plasticizer to a polyurethane foam to generate a surgical adjunct 604 described throughout. The plasticizer may be placed onto or within a surgical adjunct via bulk swelling in solution or via direct deposition to plasticize the polyurethane foam in order to reduce the foams glass transition temperature and/or reduce the mechanical strength of the polyurethane foam. In some examples, the method 1400 may optionally include adhering (optional step 1404) at least one side (e.g., bottom or crown side) of the polyurethane foam with a PDO film to create at least a partially sealed foam, subjecting (optional step 1406) the partially sealed polyurethane foam to a temperature of about 105° C. to about 115° C. for about 24 minutes, subjecting (optional step 1408) the partially seal foam to a pressure of about 5 kN, letting (optional step 1410) the partially sealed foam stand at least thirty seconds (e.g., about 60 seconds), and subjecting (optional step 1412) the partially sealed foam to a temperature of about 35° C. to about 45° C. for about 24 minutes. Step 1408 may be conducted simultaneously with steps 1406, 1410, and 1412. In some examples, surgical adjunct 604 is created without a plasticizer and the polyurethane foam is generated to have certain ratios of air to polyurethane for a given volume due to pore sizes and amounts.

As will be appreciated by one skilled in the art, The embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

In some examples, disclosed devices (e.g., end effector, surgical adjunct, and/or staple cartridges) and methods involving one or more disclosed devices may involve one or more of the following clauses:

Clause 1: A surgical adjunct 604, comprising: a polyurethane foam comprising a volumetric ratio of the polyurethane foam to the total volume of the surgical adjunct 604 is in a range of about 0.125 to about 0.325, wherein a glass transition temperature of the surgical adjunct 604 is about 0° C. to about 40° C.

Clause 2: The surgical adjunct 604 of clause 1, wherein the volumetric ratio of the polyurethane foam to total volume of the surgical adjunct is about 0.175 to about 0.225.

Clause 3: The surgical adjunct 604 of clause 1, wherein the volumetric ratio of the polyurethane foam to total volume of the surgical adjunct is about 0.19 to about 0.21

Clause 4: The surgical adjunct 604 of clause 1, wherein the glass transition temperature of the surgical adjunct is about 7.5° C. to about 22.5° C.

Clause 5: The surgical adjunct 604 of clause 1, wherein the glass transition temperature of the surgical adjunct 604 is about 12.5° C. to about 17.5° C.

Clause 6: The surgical adjunct 604 of clause 1, wherein a median pore size of the surgical adjunct 604 is about 0.025 to about 0.300 mm³.

Clause 7: The surgical adjunct 604 of clause 1, further comprising a polydioxanone (PDO) film disposed on at least one surface of the polyurethane foam.

Clause 8: The surgical adjunct 604 of clause 7, wherein the PDO film has a thickness of about 20 μm to about 100 μm.

Clause 9: The surgical adjunct 604 of claim 1, further comprising a plasticizer added to the polyurethane foam.

Clause 10: The surgical adjunct 604 of clause 9, wherein the plasticizer comprises at least one of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid.

Clause 11: The surgical adjunct 604 of clause 9, wherein the plasticizer comprises at least one fatty acid.

Clause 12: The surgical adjunct 604 of clause 1, wherein the surgical adjunct 604 has a compression strength of about 30 kPa to about 70 kPa.

Clause 13: The surgical adjunct 604 of clause 12, wherein the compression strength is about 30 to about 60 kPa.

Clause 14: The surgical adjunct 604 of clause 1, wherein the surgical adjunct 604 has a peak tensile strength of about 50 to about 150 kPa.

Clause 15: The surgical adjunct 604 of clause 1, wherein the surgical adjunct 604 has: a length of about 40 mm to about 80 mm; a width of about 8 mm to about 12 mm; and a height of about 2.5 mm to about 3.5 mm.

Clause 16: The surgical adjunct 604 of clause 15, wherein the surgical adjunct 604 has a distal end chamfer 604b and a proximal end chamfer 604c, wherein the distal end chamfer comprises a vertical portion extending from a bottom of the surgical adjunct 604, the vertical portion has a height of about 0.009 inches to about 0.029 inches, wherein the distal end chamfer comprises an angled portion extending from the vertical portion to a top surface 604a of the surgical adjunct, the angled portion has a slope of about 30 degrees to about 60 degrees in reference to the top surface, wherein the proximal end chamfer comprises a proximal end with a width of about 0.27 inches to about 0.29 inches, wherein the proximal end chamfer 604c comprises a first angled side extension and a second angled side extension extending away from the distal end of the surgical adjunct to the proximal end of proximal end chamfer 604c, the first angled side extension and the second angled side extensions each have lengths of about 0.01 inches to about 0.40 inches when measured horizontally.

Clause 17: The surgical adjunct 604 of clause 1, further comprising a plurality of struts having a median strut thickness of about 0.025 mm to about 0.300 mm.

Clause 18: A method of making a surgical adjunct 604, comprising: selectively adding a plasticizer to a polyurethane foam to generate the surgical adjunct 604, wherein the surgical adjunct 604 has a glass transition temperature of about 0° C. to about 40° C.

Clause 19: The method of clause 18, wherein selectively adding the plasticizer comprises either soaking the polyurethane foam in in a solution comprising the plasticizer or adding the plasticizer on at least a portion of at least one surface of the polyurethane foam via direct deposition, and wherein a volumetric ratio of the polyurethane foam to the total volume of the surgical adjunct 604 is in a range of about 0.125 to about 0.325.

Clause 20: The method of clause 18, further comprising: laminating the polyurethane foam with a polydioxanone (PDO) film by: adhering one side of the polyurethane foam with a PDO film to create a partially sealed foam; subjecting the partially sealed foam to a temperature of about 105° C. to about 115° C.; subjecting the partially sealed foam to a pressure of about 5 kN; and letting the partially sealed foam stand at least thirty seconds; and subjecting the partially sealed foam to a temperature of about 35° C. to about 45° C.

Clause 21: A surgical adjunct 604, comprising: a polyurethane foam, and wherein a glass transition temperature of the surgical adjunct 604 is about 0° C. to about 40° C.

Clause 22: The surgical adjunct 604 of clause 21, wherein a volumetric ratio of the polyurethane foam to the total volume of the surgical adjunct 604 is in a range of about 0.125 to about 0.325.

Clause 23: The surgical adjunct 604 of clause 21, wherein the volumetric ratio of the polyurethane foam to total volume of the surgical adjunct is about 0.175 to about 0.225.

Clause 24: The surgical adjunct 604 of clause 21, wherein the volumetric ratio of the polyurethane foam to total volume of the surgical adjunct 604 is about 0.19 to about 0.21

Clause 25: The surgical adjunct 604 of any of clauses 21 to 24, wherein the glass transition temperature of the surgical adjunct is about 7.5° C. to about 22.5° C.

Clause 26: The surgical adjunct 604 of any of clauses 21 to 24, further comprising a plasticizer added to the polyurethane foam, wherein the glass transition temperature of the surgical adjunct 604 is about 12.5° C. to about 17.5° C.

Clause 27: The surgical adjunct 604 of any of clauses 21 to 26, wherein a median pore size of the surgical adjunct 604 is about 0.025 to about 0.300 mm³.

Clause 28: The surgical adjunct 604 of any of clauses 21 to 27, further comprising a polydioxanone (PDO) film disposed on at least one surface of the polyurethane foam.

Clause 29: The surgical adjunct 604 of clause 28, wherein the PDO film has a thickness of about 20 μm to about 100 μm.

Clause 30: The surgical adjunct 604 of clause 26, wherein the plasticizer comprises at least one of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid.

Clause 31: The surgical adjunct 604 of clause 26, wherein the plasticizer comprises at least one fatty acid.

Clause 32: The surgical adjunct 604 of any of clauses 21 to 31, wherein the surgical adjunct 604 has a compression strength of about 30 to about 70 kPa.

Clause 33: The surgical adjunct 604 of any of clauses 21 to 31, wherein the surgical adjunct 604 has a compression strength is about 30 to about 50 kPa.

Clause 34: The surgical adjunct 604 of any of clauses 21 to 33, wherein the surgical adjunct 604 has a peak tensile strength of about 50 to about 150 kPa.

Clause 35: The surgical adjunct 604 of any of clauses 21 to 33, wherein the surgical adjunct 604 has a peak tensile strength of about 45 to about 85 kPa.

Clause 36: The surgical adjunct 604 of any of clauses 21 to 35, wherein the surgical adjunct 604 has: a length of about 40 mm to about 80 mm; a width of about 8 mm to about 12 mm; and a height of about 2.5 mm to about 3.5 mm.

Clause 37: The surgical adjunct 604 of any of clauses 21 to 35, wherein the surgical adjunct 604 has: a length of about 60 mm to about 70 mm or about 45 mm to about 55 mm; a width of about 9.75 mm to about 10.25 mm; and a height of about 2.85 mm to about 3.15 mm.

Clause 38: The surgical adjunct 604 of any of clauses 21 to -35, wherein the surgical adjunct 604 has: a length of about 66.04 mm to about 66.3 mm or about 21.12 mm to about 51.38 mm; a width of about 10.025 mm to about 10.035 mm; and a height of about 2.95 mm to about 3.05 mm.

Clause 39: A surgical staple cartridge assembly 106, comprising: a cartridge 200 having a length of about 80 mm to about 90 mm and a width of about 8.9 mm to about 14 mm; a surgical adjunct 604 disposed on the cartridge 200 comprising: a polyurethane foam comprising a volumetric ratio of the polyurethane foam to the total volume of the surgical adjunct 604 is in a range of about 0.125 to about 0.325, wherein a glass transition T_{g} temperature of the surgical adjunct 604 is about 0° C. to about 40° C., and wherein the surgical adjunct 604 having: a length of about 40 mm to about 80 mm; a width of about 8 mm to about 12 mm; and a height of about 2.5 mm to about 3.5 mm.

Clause 40: The surgical cartridge assembly 106 of clause 39, wherein the surgical adjunct 604 further comprises a plasticizer added to the polyurethane foam.

Clause 41: The surgical cartridge assembly 106 of clauses 39 or 40, wherein the cartridge 200 comprises at least two approximately parallel sets of staple cavities 212, 214 spaced apart by a longitudinal slot 210, each staple cavity 212, 214 having: an approximately parallel orientation with the longitudinal slot 210; a mouth-like shape; a maximum length of about 0.122 to about 0.124 inches; and a maximum width of about 0.023 inches to about 0.027 inches.

Clause 42: The surgical cartridge assembly 106 of clause 39, wherein at least centers of two adjacent staple cavities 212 in a row 212a are spaced apart by about 0.158 inches.

Clause 43: The surgical cartridge assembly 106 of clause 38, wherein the surgical adjunct 604 comprises two parallel portions 806a, 806b separated by a slot 808.

Clause 44: The surgical cartridge assembly 106 of clause 38, wherein the surgical adjunct 604 comprises two parallel portions 806a, 806b separated by at least one slit 808, at least one bridge 802 connecting the two parallel portions 806a, 806b across the at least one slit 808.

Clause 45: The surgical cartridge assembly 106 of clause 44, wherein the at least one bridge 802 comprises a length of about 0.035 inches to about 0.046 inches.

Clause 46. The surgical cartridge assembly 106 of any of clauses 39 to 45, wherein an adhesive is disposed between the surgical adjunct 604 and the cartridge 200 and is configured to adhere the surgical adjunct to the cartridge.

Clause 47: The surgical cartridge assembly 106 of clause 46, further comprising about 100 mg to about 120 mg of adhesive between the cartridge 200 and the surgical adjunct 604.

Clause 48: The surgical cartridge assembly 106 of any of clauses 39 to 47, wherein the cartridge 200 comprises: a deck 206 comprising at least one raised surface 804 along lateral and/or distal sides of the cartridge 200 and configured to align the surgical adjunct 604; and an atraumatic shaped distal end DC.

Clause 49: The surgical cartridge assembly 106 of any of clauses 39 to 47, wherein the cartridge 200 comprises a substantially flat deck 206.

Clause 50: The surgical cartridge assembly 106 of any of clauses 39 to 48, wherein the cartridge 200 comprises a plurality of raised surfaces positioned at each distal end and proximal end of each staple cavity 212, 214, and wherein a raised surface corresponding to a distal end of a staple cavity 212, 214 forms a combined raised surface with a raised surface corresponding to a proximal end of an adjacent staple cavity 212, 214.

Clause 51: The surgical cartridge assembly 106 of any of clauses 39 to 50, wherein the cartridge 200 comprises a plurality of lowered surfaces that are lower than the deck 206, and wherein at least one lowered surface is positioned along at least one lateral side of a staple cavity 212, 214.

1-15. (canceled)

16. A surgical adjunct, comprising:
a polyurethane foam comprising a volumetric ratio of the polyurethane foam to the total volume of the surgical adjunct is in a range of about 0.125 to about 0.325,
wherein a glass transition temperature of the surgical adjunct is about 0° C. to about 40° C.

17. The surgical adjunct of claim 16, wherein the volumetric ratio of the polyurethane foam to total volume of the surgical adjunct is about 0.175 to about 0.225.

18. The surgical adjunct of claim 16, wherein the volumetric ratio of the polyurethane foam to total volume of the surgical adjunct is about 0.19 to about 0.21

19. The surgical adjunct of claim 16, wherein the glass transition temperature of the surgical adjunct is about 7.5° C. to about 22.5° C.

20. The surgical adjunct of claim 16, wherein the glass transition temperature of the surgical adjunct is about 12.5° C. to about 17.5° C.

21. The surgical adjunct of claim 16, wherein a median pore size of the surgical adjunct is about 0.025 to about 0.300 mm³.

22. The surgical adjunct of claim 16, further comprising a polydioxanone (PDO) film disposed on at least one surface of the polyurethane foam.

23. The surgical adjunct of claim 22, wherein the PDO film has a thickness of about 20 μm to about 100 μm.

24. The surgical adjunct of claim 16, further comprising a plasticizer added to the polyurethane foam.

25. The surgical adjunct of claim 24, wherein the plasticizer comprises at least one of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid.

26. The surgical adjunct of claim 24, wherein the plasticizer comprises at least one fatty acid.

27. The surgical adjunct of claim 16, wherein the surgical adjunct has a compression strength of about 30 kPa to about 70 kPa.

28. The surgical adjunct of claim 27, wherein the compression strength is about 30 to about 60 kPa.

29. The surgical adjunct of claim 16, wherein the surgical adjunct has a peak tensile strength of about 50 to about 150 kPa.

30. The surgical adjunct of claim 16, wherein the surgical adjunct has: a length of about 40 mm to about 80 mm; a width of about 8 mm to about 12 mm; and a height of about 2.5 mm to about 3.5 mm.

31. The surgical adjunct of claim 30, wherein the surgical adjunct has a distal end chamfer and a proximal end chamfer,
wherein the distal end chamfer comprises a vertical portion extending from a bottom of the surgical adjunct, the vertical portion has a height of about 0.009 inches to about 0.029 inches,
wherein the distal end chamfer comprises an angled portion extending from the vertical portion to a top surface a of the surgical adjunct, the angled portion has a slope of about 30 degrees to about 60 degrees in reference to the top surface,
wherein the proximal end chamfer comprises a proximal end with a width of about 0.27 inches to about 0.29 inches, and
wherein the proximal end chamfer comprises a first angled side extension and a second angled side extension extending away from the distal end of the surgical adjunct to the proximal end of proximal end chamfer, the first angled side extension and the second angled side extensions each have lengths of about 0.01 inches to about 0.40 inches when measured horizontally.

32. The surgical adjunct of claim 1, further comprising a plurality of struts having a median strut thickness of about 0.025 mm to about 0.300 mm.

33. A method of making a surgical adjunct, comprising:
selectively adding a plasticizer to a polyurethane foam to generate the surgical adjunct,
wherein the surgical adjunct has a glass transition temperature of about 0° C. to about 40° C.

34. The method of claim 33, wherein selectively adding the plasticizer comprises either soaking the polyurethane foam in in a solution comprising the plasticizer or adding the plasticizer on at least a portion of at least one surface of the polyurethane foam via direct deposition, and
wherein a volumetric ratio of the polyurethane foam to the total volume of the surgical adjunct is in a range of about 0.125 to about 0.325.

35. The method of claim 33, further comprising:
laminating the polyurethane foam with a polydioxanone (PDO) film by:
adhering one side of the polyurethane foam with a PDO film to create a partially sealed foam;
subjecting the partially sealed foam to a temperature of about 105° C. to about 115° C.;
subjecting the partially sealed foam to a pressure of about 5 kN;
letting the partially sealed foam stand at least thirty seconds; and
subjecting the partially sealed foam to a temperature of about 35° C. to about 45° C.
